(19) United States
(12) Patent Application Publication
(10) Pub. No.: US 2025/0173887 A1
(43) Pub. Date: May 29, 2025

(54) METHOD FOR MEASURING OIL DISPERSION CAPACITY ON WATER SURFACE

(71) Applicants: PETRÓLEO BRASILEIRO S.A. – PETROBRAS, Rio de Janeiro (BR); UNIVERSIDADE FEDERAL DO RIO DE JANEIRO - UFRJ, Rio de Janeiro (BR)

(72) Inventors: MARCO ANTÔNIO GOMES TEIXEIRA, Niterói (BR); ANA MEHL, Rio de Janeiro (BR); LEANDRO AUGUSTO FRATA FERNANDES, Niterói (BR); ROGERIO MESQUITA DE CARVALHO, Rio de Janeiro (BR); THIAGO ANDRADE DELFINO DA SILVA, Rio de Janeiro (BR)

(73) Assignees: PETRÓLEO BRASILEIRO S.A. – PETROBRAS, Rio de Janeiro (BR); UNIVERSIDADE FEDERAL DO RIO DE JANEIRO - UFRJ, Rio de Janeiro (BR)

(21) Appl. No.: 18/768,961

(22) Filed: Jul. 10, 2024

(30) Foreign Application Priority Data
Aug. 28, 2023 (BR) 1020230172954

(51) Int. Cl. G06T 7/62; G06T 7/11; G06T 7/136; G06T 7/187

(52) U.S. Cl. CPC G06T 7/62; G06T 7/11; G06T 7/136; G06T 7/187; G06T 2207/10016

(57) ABSTRACT

The present invention relates to a method for measuring the oil dispersion capacity on a water surface, a system for releasing oil and computer-readable storage media. More specifically, the present invention relates to a method for measuring the oil dispersion capacity on a water surface, by capturing videos of the formation of an oily spot on a water surface.

### CROSS-REFERENCE TO RELATED APPLICATION

This application claims priority to Brazil patent application Ser. No. 10/202,30172954, filed on Aug. 28, 2023, the contents of which are hereby incorporated by reference in their entireties for all purposes.

### FIELD OF THE INVENTION

The present invention falls within the technical field of primary processing technologies, more specifically in the technical field of environmental monitoring and recovery. In particular, the present invention relates to a method for measuring the oil dispersion capacity on a water surface, a system for releasing oil and computer-readable storage media.

### BACKGROUND OF THE INVENTION

In oil production, which involves the separation

of water and oil, the destination of this water is generally disposal. Considering oil processing operations in an offshore environment, the disposal of water, which occurs at sea, is regulated by legislation with regard to the concentration of oil in water, which is commonly referred to in Brazil as oil and grease content (TOG).

Depending on some conditions, the presence of oil in the sea can result in the formation of oily features or oily spots on the sea surface. The ability of these features to form on the sea surface is supposedly associated with the concentration of oil present in the discarded produced water.

Although several conditions can influence the formation of oily features on the sea surface (for example, wind conditions, temperature, sea, among others), it is clear that the higher the oil concentration, the greater the probability of formation of oily features.

However, the variation in oil concentration depending on the type of oil is unknown. As is also known, there are oils with different characteristics and compositions. In this sense, the impact and relationship of these differences associated with TOG values in discharged water is unknown.

Therefore, many doubts are raised about the real impact of water disposal and the formation of oily features, making it possible to make decisions regarding the reduction of the TOG value in water disposal or possible process flexibility.

Currently, on a laboratory scale and under controlled conditions, the most commonly adopted approach is static testing (Static Sheen Test—EPA 1617). This test consists of dispersing oil in a controlled manner in a prepared large container. The container is left to rest for approximately 1 hour, when oily spot formation is observed. This test is basically applied to drilling fluids and other working fluids on offshore platforms, but from an environmental point of view, the formation of an oil spot is not accepted after this period. If an oil spot is formed, the use of this type of fluid in an offshore environment is not permitted. This test is commonly sold in the United States of America and can be applied to petroleum. However, it is a qualitative test, not allowing for a more critical evaluation.

Therefore, there is a clear lack of a solution that allows the evaluation of different types of oils and their respective potential for the formation of oily features.

Furthermore, the need for a solution that allows understanding the differences and participation of a certain component present in the oil in the formation of oily features is evident, allowing the planning of actions to mitigate or minimize the risks of formation of these features, such as actions to reduce the TOG values present in the water or restrict the production of a certain stream that presents a greater possibility of the formation of oily features.

### STATE OF THE ART

In the state of the art, there are solutions that detect the presence of oil on the seawater surface through an image, as will be discussed below.

Document BR 112015003444-6 describes a method and system involving machine learning to detect and evaluate the presence of oil on the seawater surface, through an image. In particular, this document addresses a computer vision engine configured to segment image data into detected spots or bubbles of surface oil. The processed images are not visible light images, but images from three or more long wavelength infrared (LWIR) cameras, whose outputs are filtered by different band pass filters, and the signals are multiplexed to generate a single synthetic signal, the brightness of which indicates a similarity to an oil signature on the sea surface. The present invention differs from document BR 112015003444-6, as it uses video that is captured in visible light, with colors encoded in the RGB space.

Furthermore, the document Durve, M., Bonaccorso, F., Montessori, A. et al. Tracking droplets in soft granular flows with deep learning techniques. Eur. Phys. J. Plus 136, 864 (2021), accessible at https://doi.org/10.1140/epjp/s13360-021-01849-3, describes a method for object recognition in binary images generated synthetically by gluing ellipses onto a black background of fluids, more specifically, of droplets of a flow moving on the surface, using deep learning techniques. In contrast, in the present invention, the droplets are detected in true color images (RGB) and are sometimes on an upward trajectory in the medium and sometimes on the surface before falling apart, thus the detection and tracking mechanisms are prepared to consider partial or total occlusion, which occurs when a superficial droplet blocks the view of the camera of an upward droplet.

### BRIEF DESCRIPTION OF THE INVENTION

The present invention defines, according to a preferred embodiment thereof, a method for measuring the oil dispersion capacity on a water surface, comprising the steps of: capturing at least one video; processing the video, which comprises identifying at least one frame-by-frame element of the video, independently between frames; wherein the at least one element is identified by at least one bounding box; further including the step of detecting droplet, wherein the element is detected as a droplet candidate element or an element classified as a droplet; estimating the motion flow, which comprises creating an intermediate frame t+1 from two frames of the video captured at time instants t and t+2; and creating two motion flow maps associated with the intermediate frame at time instant t+1, wherein the motion flow maps include vectors in the two-dimensional space of the image; wherein the first motion flow map indicates where the frame pixels at time instant t went at time instant t+1 and the second motion flow map indicates where the frame pixels at time instant t+2 came from the intermediate frame at time instant t+1; and wherein the motion flow maps maintain the same resolution in pixels as the video frames at time instants t and t+2 and assign to each pixel of the frame at time instant t+1 the identifying number of the droplet that gave rise to the oil present in that pixel, wherein the identifying number is an integer value greater than or equal to 1, or the value 0 if the pixel is not associated with the oil of any droplet; and wherein the estimate of the percentage of spreading flow is given by dividing the number of pixels associated with a droplet by the total number of pixels in the image, 100 times; tracking the droplet and the spreading of the oil involves defining that the pixels in the frame considered as the origin of the oil from a droplet that dissolves on the water surface are those that correspond to the area occupied by the droplet in the last update made by it in the drop candidate list, before its promotion to the second list of classified as droplets; wherein the oil area for each droplet in pixels is given by counting the number of pixels that contain the droplet identifier on the map in question; and wherein the relative area is given by the area of the oil in pixels divided by the total area of the image; and obtaining the processing results, which includes obtaining the captured video, a video with detected droplets, and tracked droplets and spreading; and indication, for each detected droplet, of the moment relative to the beginning of the video wherein the beginning of its formation was detected, the moment wherein the droplet disintegrates on the water surface and the relative area occupied by the oil of that droplet every t seconds of the video during spreading.

The at least one video is captured by at least one capture device comprising at least one camera.

In the step of video processing, a bounding box is discarded when the detection confidence is lower than a temporal threshold t_{C}.

In the step of video processing, the Jaccard coefficient is used to identify all bounding boxes that delimit the same element, wherein the intersection area of a pair of bounding boxes divided by the union area of the same bounding boxes must be greater than a Jaccard threshold t_{J}.

The box most likely to enclose the same element is selected by applying the non-maximum suppression process.

The identified element is classified as any of a new droplet of oil, a known droplet of oil that is on its way to the surface, a known droplet of oil that is on the surface, an air bubble, or a spurious element.

An identified element is detected as a droplet candidate and placed in a first droplet candidate list when the element is not associated with any element already in the first droplet candidate list or in a second list of classified as a droplet.

The element is inserted into the first droplet candidate list with the bounding box of this element and the video frame number where the first identification of this element occurred.

An element is identified as previously included in the first droplet candidate list, when the bounding box of this element is equal to and overlaps with the bounding box of an element included in the first droplet candidate list; and wherein the element is included in the first droplet candidate list with the new bounding box information and video frame number where the updated information was collected.

An element is identified as previously included in the second list of classified as a droplet, when the bounding box of this element has a size equal to and overlaps with the bounding box of an element included in the second list of element classified as a droplet; and wherein the element is included in the second list of classified as a droplet with the new bounding box information and video frame number where the updated information was collected.

If an element remains for more than t_{P} seconds in the first droplet candidate list without updating, the element is discarded from the first droplet candidate list; or if it is identified that the total lifetime of the element from the first and last detection of the element is greater than t_{L} seconds, then the element moves to the second list of classified as a droplet.

Furthermore, according to another preferred embodiment of the present invention, it defines a system for releasing oil, comprising: an oleophilic crucible with a sheet of plastic material with a depression to contain oil; a camera with a mirror; and a video capture device, wherein the video capture device is positioned parallel to the water surface, fixed on a base; wherein the video captured by the video capture device is processed in accordance with the method for measuring the oil dispersion capacity on a water surface. The chamber is made of glass and has two measuring scales. The mirror comprises a physical indication for calibration.

Additionally, according to a preferred embodiment, the present invention relates to a system for releasing oil comprising: a container with a septum, wherein the container is oriented so that the septum is arranged with its inlet down; a hollow needle, wherein the hollow needle is inserted into the septum; a video capture device, wherein the video capture device is movably arranged on the top of the container; and a measuring scale; wherein the video captured by the video capture device is processed in accordance with the method for measuring the oil dispersion capacity on a water surface. The video capture device is movably arranged on the top of the container (100) via a support.

Furthermore, according to a preferred embodiment, the present invention relates to a computer-readable storage medium comprising stored therein, a set of computer-readable instructions, which when executed by one or more computers, the one or more computers carry out the method for measuring the oil dispersion capacity on the water surface.

### BRIEF DESCRIPTION OF THE FIGURES

In order to complement the present description and obtain a better understanding of the features of the present invention, and in accordance with a preferred embodiment thereof, in annex, a set of figures is submitted, where in an exemplified, although not limiting, manner, represents the preferred embodiment.

FIG. 1 represents a video resulting from video processing by the method of the present invention.

FIG. 2 illustrates the flowchart of the method of the present invention.

FIG. 3A exhibits an image showing an experiment with an air bubble, which is correctly not detected as a droplet.

FIG. 3B exhibits an image showing the experiment with a droplet at the moment exactly before the oil expands on the surface.

FIG. 3C exhibits an image showing the beginning of the oil spreading, with consequent breakup of the droplet.

FIG. 3D exhibits an image showing the spreading of the oil, still indicating the location of the original droplet.

FIG. 3E exhibits an image showing a homogeneous spot, indicating an advanced stage of oil spreading.

FIG. 3F exhibits an image showing a surface with the presence of oil with spreading close to total.

FIG. 4A exhibits an image showing an experiment with an air bubble, which is correctly not detected as a droplet, with the method of the present invention.

FIG. 4B exhibits an image showing an experiment with a droplet at the moment exactly before expanding the oil on the surface, with the method of the present invention.

FIG. 4C exhibits an image showing the beginning of the oil spreading, with consequent breakup of the droplet, with the method of the present invention.

FIG. 4D exhibits an image showing the spreading of the oil, still indicating the location of the original droplet, with the method of the present invention.

FIG. 4E exhibits an image showing a homogeneous spot, indicating an advanced stage of oil spreading, with the method of the present invention.

FIG. 5A shows the detection of a first oil droplet.

FIG. 5B shows the detection of a second oil droplet.

FIG. 5C shows the detection of a third oil droplet.

FIG. 5D shows the detection of a fourth oil droplet.

FIG. 5E shows the detection of a fourth oil droplet.

FIG. 5F shows the detection of a fourth oil droplet.

FIG. 6 illustrates the phenomenon of saturation of the water surface by oil from the droplets.

FIG. 7A shows image processing after saturating the surface with 7 droplets.

FIG. 7B shows image processing after saturating the surface with 9 droplets.

FIG. 8 displays a graph with the percentage variation in the area of the processed video.

FIG. 9 shows an oleophilic crucible.

FIG. 10 shows an oleophilic crucible and the chamber with mirror.

FIG. 11A shows the crucible filled with oil.

FIG. 11B shows the moment of release of the droplet from the edge.

FIG. 11C shows the droplet reaching the surface.

FIG. 11D shows the moment when the oil began to spread on the surface.

FIG. 11E shows the release of a new droplet.

FIG. 12A shows the moment of release of the droplet from the edge of the crucible.

FIG. 12B shows the moment when the released droplet hits the surface.

FIG. 13 shows a hollow needle.

FIG. 14 shows a container with an inlet with a rubber septum.

FIG. 15 represents the oil release system of the present invention.

FIG. 16 is an image captured by the capture device.

FIG. 17 shows another variation in the height of the needle tip inserted into the container.

FIG. 18 shows the release of an oil droplet into the seawater from the needle tip.

FIG. 19 illustrates a droplet that reaches the surface and spreads.

FIG. 20 illustrates a new released droplet.

### DETAILED DESCRIPTION OF THE INVENTION

The method for measuring the dispersion capacity of oils on a water surface and the system for releasing oil, according to a preferred embodiment of the present invention, are described in detail below, based on the attached figures.

The present invention relates to a method for measuring the oil dispersion capacity on a water surface, by capturing and recording videos of the formation of an oily spot on a water surface.

Furthermore, the present invention relates to a system for releasing oil.

According to a preferred embodiment, the method for measuring the oil dispersion capacity on a water surface can be implemented through a set of instructions readable by a computer or processor or machine, wherein the set of instructions is executed by one or more computers or processors or machines, wherein the set of instruction may be stored or recorded on a storage medium readable by the computer or processor or machine. The processing of the set of instructions that perform the method for measuring the oil dispersion capacity on a water surface of the present invention can be executed via CPU or using a GPU or TPU for better computational performance. Otherwise, the set of computer-readable instructions represents a computer program or application.

According to a preferred embodiment of the present invention, the way the oil will spread or the way the images will be captured do not affect the processing of the images.

According to a preferred embodiment of the present invention, an artificial neural network is used to detect an oil droplet, from its formation to its disappearance, at the moment the droplet disintegrates, spreading the oil on the water surface.

For example, the oil droplet can be injected into the bottom of a container with water, for example.

The artificial neural network for detecting oil droplets, together with a library implemented for the method for measuring the oil dispersion capacity on a water surface of the present invention, was specialized for the detection of oil droplets, since that conventional detection networks are trained to detect and classify other types of objects present in traditional image bases, such as the coco network.

Regarding the identification of elements, this occurs frame by frame of the video, independently between frames. Especially, the library implemented for the method for measuring the oil dispersion capacity on a water surface, according to the present invention, uses its own heuristics to suppress false detections, such as, for example, the detection of air bubbles, and to perform droplet tracking between frames.

As an example, when a droplet breaks on the surface of water, the oil contained in this droplet spreads. In the library developed for the method for measuring the oil dispersion capacity on a water surface, the spreading flow is estimated by an artificial neural network, initially developed to create intermediate frames in videos with low frame rates per second.

In particular, the artificial neural network used as a basis was pre-trained by its authors with Hollywood films. Therefore, the parameterization of this network was necessary to meet the needs of the present invention, since videos of oil spots have different features from the videos originally used in the original artificial neural network used as a base.

With droplet spreading tracking, the library developed for the method for measuring the oil dispersion capacity on a water surface, estimates, as a function of time, the percentage of the video frame area that is covered by the oil contained in each droplet.

Regarding identifying the video element frame by frame independently between frames, note that for now the oil droplet is called an “element” because the identified element may not be a droplet (for example, may be an air bubble), requiring the use of heuristics for classification.

Furthermore, before treatment, an element can be identified more than once, as the artificial neural network used returns, as a raw detection result, a set of bounding boxes. Therefore, multiple reported bounding boxes may overlap, making it necessary to choose the bounding box that best delimits an element.

A bounding box is discarded when the detection confidence is lower than a temporal threshold t_{C}.

For the remaining bounding boxes, the Jaccard coefficient is used to collect all bounding boxes that potentially delimit the same element. In other words, the area of intersection of a pair of bounding boxes divided by the area of their union must be greater than a Jaccard threshold ty for the overlap to be considered significant, that is, the bounding boxes are delimiting the same element.

In particular, from the overlapping bounding boxes associated with the same element, the most probable box is selected by applying the non-maximum suppression process.

Specifically, an identified element can be classified as any of a new oil droplet, a known oil droplet that is on its way to the surface, a known oil droplet that is on the surface, an air bubble or some spurious element.

To identify which of the cases the identified element fits into and classify it as one of the elements indicated above, it is necessary to analyze the history of elements tracked in previous video frames. As a consequence, the heuristics for detecting new droplets, the heuristics for tracking droplets between frames and the heuristics for detecting air bubbles are interconnected.

Regarding the history of identified elements, it is important to comment that according to the proposed method, two lists are maintained. In a first list, the element that is a candidate for the droplet is stored. In the second list, the element classified as a droplet is stored.

An identified element is classified as a droplet candidate and inserted into the first droplet candidate list whenever it is not associated with any element already in one of the first list or second list. When this element is inserted into the first droplet candidate list, the bounding box of this element and the video frame number where the first identification of the droplet candidate element occurred are stored.

To be considered an element previously included in one of the first list or second list, the bounding box of this element must be of similar size and sufficient overlap with the bounding box of a droplet candidate element (included in the first droplet candidate list) or an element already classified as droplet (contained in the second list of elements classified as droplet). If this is the case, that is, the element is identified by already being included in one of the first list or second list, the respective list where the element was already included is updated with the new bounding box information and video frame number where the updated information was collected. The definition of “similar size or sufficient overlap” is associated with the captured area of the image and the response obtained. That is, the distance between the capture element and the water surface.

If an element remains for more than t_{P} seconds in the first droplet candidate list without updating, then that element is discarded from the first droplet candidate list. But if, simultaneously, it is identified that the total lifetime of the element (that is, the time elapsed between the first and last detection of the element) is greater than t_{L} seconds, then the element is included in the second list of classified as a droplet.

Specifically, in processed videos, air bubbles are often discarded by the t_{P} threshold. However, because they rise quickly to the surface, air bubbles are not promoted to droplets by the t_{L} threshold. This applies to other spurious elements, as they have a very short lifetime as they are outliers in the detection process.

t_{P} and t_{L} thresholds, mentioned above, can be configurable. t_{P} and t_{L} values used in the experiments were found through observation of the videos provided. The configuration of t_{P} and t_{L} thresholds will depend on the oil injection flow rate and the desired interval between droplet placement, generally varying from around 1 to 10 seconds.

In the developed library, the spreading flow is estimated by an artificial neural network, initially developed for creating intermediate frames in videos with low frames per second rate. In other words, the artificial neural network receives as inputs two frames of the video, captured at time t and t+1, and produces an artificial frame with the estimated content for time t+½. In addition to the estimated video frame, the artificial neural network also produces two vector maps. The first vector map indicates where the pixels in the frame at time t went at time t+½. The second vector map indicates where the pixels of the frame at time t+1 came from in the estimated frame at time t+½.

These maps are known in the literature as motion flow maps.

In the developed library, instead of reporting consecutive frames of the video (that is, captured at time t and t+1), frames captured at time t and t+2 are reported. Therefore, the motion flow maps produced in the present invention are associated in an existing t+1 frame. The portions of these motion flow maps related to the oil on the water surface correspond to the oil droplet spreading estimate.

By tracking the spreading of the droplet, the developed library estimates, as a function of time, the percentage of area of the video frame that is covered by the oil contained in each droplet.

The parameterization according to the present invention deals with the resolution of the input frames and the motion flow maps issued at the output. In fact, pre-processing is carried out to adapt.

In the case of droplet detection, the artificial neural network used is YOLOv5, which receives as input an RGB (red, green, and blue) color image and outputs bounding boxes for the detected elements as output.

The YOLOv5 artificial neural network was pre-trained by its authors with the coco dataset for all classes predicted in this dataset, and was then specialized for the “droplet” class with a dataset created from videos of oily features.

The hyperparameters used are the same as those considered in pre-training with coco, carried out by the authors of YOLOv5. Details are provided in the source repository: https://github.com/ultralytics/yolov5.

In the case of spreading flow estimation, the network used is RIFE HD v3, which receives as input a pair of consecutive color frames (RGB) and produces as output an intermediate color frame (RGB) and two flow maps containing vectors in the two-dimensional space of the image. The model was pre-trained by its authors with the Vimeo90K dataset. The hyperparameters used were defined by the RIFE authors. Details are provided in the source repository: https://github.com/megvii-research/ECCV2022-RIFE.

The library maintains a map with the same resolution as the video frames. This map assigns to each pixel of the current frame the identifying number of the droplet that gave rise to the oil present in that pixel (integer value greater than or equal to 1), or the value 0 if the pixel is not associated with the oil of any droplet. The estimate of the spreading flow percentage is given by dividing the number of pixels associated with a droplet by the total number of pixels in the image, 100 times.

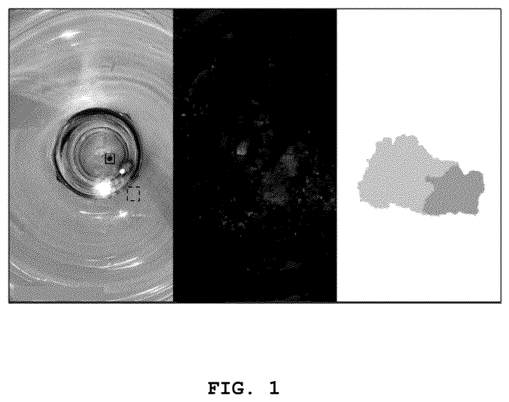

The video generated as output by the implemented library exhibits three views, as shown in FIG. 1.

FIG. 1 shows images displayed by the video

generated as a result of video processing, according to an embodiment of the method for measuring the oil dispersion capacity on a water surface of the present invention.

In the first view, shown on the left of FIG. 1, it represents the current frame of the processed video, wherein the rectangle in continuous line corresponds to the bounding box of the element detected and classified as a droplet, but which has not yet spread oil on the water surface; and the rectangle in dashed line corresponds to the detected element, but which did not go beyond droplet candidate status.

In the second view, in the center of FIG. 1, the estimated motion flow map view for the current frame is displayed. The brightness of the colors in this visualization (V channel in the HSV color space) represents the flow velocity at a given pixel (that is, information associated with the magnitude of the flow vector). The greater the magnitude, the brighter the color. The hue (H channel in the HSV color space) represents the direction of the flow vector, encoded by angles.

The third view, shown on the right of FIG. 1, exhibits the map that associates each pixel of the frame coming from the processed video with the oil from a droplet that has already spread on the water surface. The oil in each droplet is assigned a random color in this display.

Video processing to produce the bounding box marks displayed in the first view, on the left of FIG. 1, occurs as explained below, as already indicated above. Element detection is performed frame by frame of the video, independently between frames, note that for now the oil droplet is called an “element”, because the detected element may not be a droplet (for example, it may be an air bubble). Furthermore, multiple reported bounding boxes may overlap, making it necessary to choose the bounding box that best delimits an element. A detected bounding box is discarded when the detection confidence is lower than a threshold t_{C}. For the remaining bounding boxes, the Jaccard coefficient is used to collect all those that potentially delimit the same element. In other words, the area of intersection of a pair of bounding boxes divided by the area of their union must be greater than a threshold ty for the overlap to be considered significant. From the overlapping bounding boxes associated with the same element, the most likely box is selected by applying the non-maximum suppression process. In particular, a reported element can be classified as any of a new oil droplet, a known oil droplet that is on its way to the surface, a known oil droplet that is on the surface, an air bubble or some spurious element. To identify which of the cases the reported element fits into, it is necessary to analyze the history of elements tracked in previous frames of the video. Regarding the history of tracked elements, two lists are maintained. In a first list, the elements that are candidates for the droplet are stored. In the second list, elements classified as droplet are stored. A detected element is inserted into the first list of droplet candidates whenever it is not associated with any element already in one of the first or second lists. When this element is inserted into the first droplet candidate list, the bounding box of this element and the video frame number where the first identification of the droplet candidate element occurred are stored. To be considered a previously known element in one of the first list or second list, the bounding box of this element must be of similar size and sufficient overlap with the bounding box of a droplet candidate element (contained in the first droplet candidate list) or of an element already classified as a droplet (contained in the second list of elements classified as a droplet). If this is the case, the list is updated with the new bounding box information and video frame number where the updated information was collected. If an element remains in the droplet candidate list for more than t_{P} seconds without updating, then that candidate is discarded. But if, simultaneously, it is identified that the total lifetime of the element (that is, the time elapsed between the first and last detection of the element) is greater than t_{L} seconds, then the element moves to the second list classified as a droplet.

The processing to produce the motion flow map, displayed in the central view of FIG. 1, occurs as described above and again explained below. The artificial neural network originally used as a base receives as input two video frames, captured at times t and t+1, and produces an artificial frame with the content estimated at time t+½. In addition to the estimated video frame, the artificial neural network also produces two vector maps. The first vector map indicates where the pixels in the frame at time t went at time t+½. The second vector map indicates where the pixels of the frame at time t+1 came from in the estimated frame at time t+½. Instead of reporting consecutive frames of the video (that is, captured at time t and t+1), according to the method of the present invention, frames captured at time t and t+2 are reported. Therefore, the motion flow maps produced in the present invention are associated in an existing t+1 frame. The portions of these motion flow maps related to the oil present on the water surface correspond to the oil droplet spreading estimate.

The processing that leads to the production of the third view, on the right of FIG. 1, is carried out as described below. The pixels in the frame considered as the origin of the oil of a droplet that dissolves on the water surface are those that correspond to the area occupied by the droplet in the last update made by it in the droplet candidate list, before its promotion to the second list of classified as droplets. Therefore, the starting moment is given by this table.

The oil area of each broken droplet is updated frame by frame with the aid of motion flow estimation. This occurs by updating the map that assigns to each pixel of the frame the identifying number of the droplet that gave rise to the oil present in that pixel. That is, if the motion flow map indicates that the content of a pixel with oil at time t has passed to another pixel at time t+1, then the droplet identifier associated with the source pixel is copied to the destination pixel in the map mentioned above.

The area of oil for each droplet (in pixels) is given by counting the number of pixels that contain the droplet identifier on the map in question. The relative area is given by the area of the oil in pixels divided by the total area of the image.

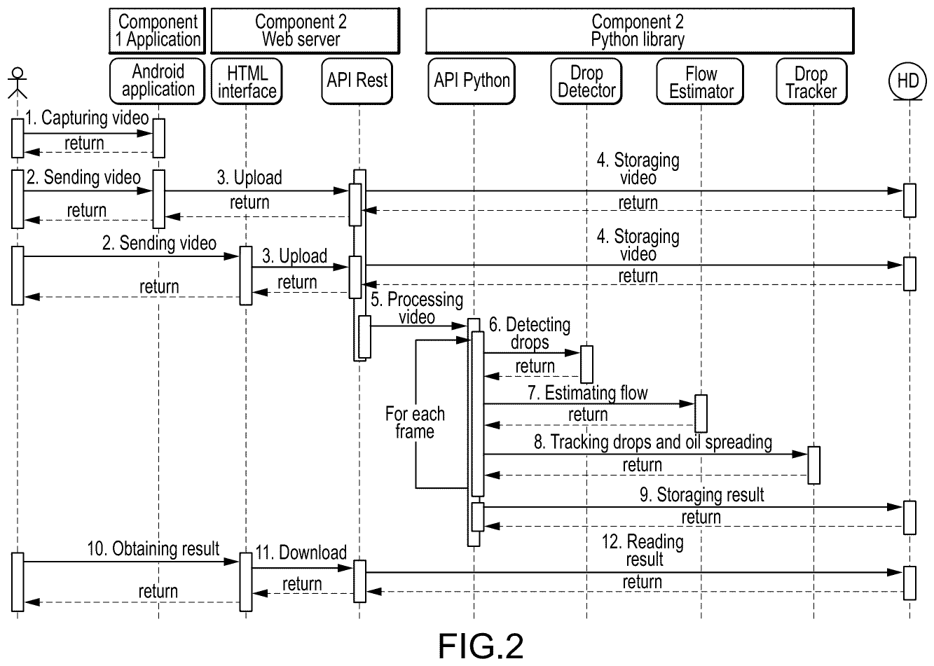

The method for measuring the oil dispersion capacity on a water surface comprises steps that are illustrated in the flowchart in FIG. 2.

The method for measuring the oil dispersion capacity on the water surface comprises the following steps: (a) capturing video; (b) uploading and processing video; and (c) obtaining the processing results, which are described in detail below.

### (a) Capturing Video 1

The user, represented in the first column on the left of FIG. 2, captures a video 1 using the application installed on a capture device.

The computer application or program represents a set of computer- or processor- or machine-readable instructions that may be stored or recorded on a computer- or processor- or machine-readable storage medium; wherein when the set of instructions is executed by one or more computers or processors or machines, the one or more computers or processors or machines performs the method for measuring the oil dispersion capacity on a water surface in accordance with the present invention.

Furthermore, the application is configured to save the video in MP4 format, for example, or any other multimedia file storage format, with the highest resolution available on the capture device.

The capture device may be any capture device that comprises at least one camera or that may include at least one attached camera. The capture device can be anything from a cell phone, a smartphone, a tablet, among others.

### (b) Sending the Video for Processing 2

The video can be sent for processing 2, by the user, either from the application or via the web interface using a personal computer. Both interfaces trigger the Rest API of the web server to upload the video 3 which, consequently, is stored 4 on the hard drive (HD) of the web server.

Activation of the Rest API, as indicated in FIG. 2, is performed by the web server. This web server exhibits an HTML interface that allows the user to select a video file on their machine (computer or smartphone, for example) that will be submitted to the server (video upload operation). In addition to accessing the web server from a browser, the user can also access it from the application that performs the steps of the method for measuring the oil dispersion capacity on the water surface. The interface of this application includes two main options: the first option allows the user to use the camera of the smartphone to record videos and save them in a gallery on the device itself; and the second option allows to inspect the gallery and submit the captured videos for processing on the web server.

After storing video 4, processing of video 5 begins asynchronously.

In the step of processing video 5, each frame of the video goes through the steps of detecting droplet 6, estimating motion flow 7, and tracking droplet and oil spreading 8.

Specifically, during the step of processing video 5, the collected oil droplet and oil spot detection and tracking data are stored 9 in memory.

### Droplet Detection 6

The step of processing video 5 is described above in this document but is reproduced below for ready reference and coherence. The step of processing video 5 comprises identifying at least one frame-by-frame element of the video, independently between frames; wherein the at least one element is identified by at least one bounding box; further including the step of detecting droplet 6, wherein the element is detected as a droplet candidate element, or an element classified as a droplet.

Furthermore, in step of processing video 5, a bounding box is discarded when the detection confidence is lower than a temporal threshold t_{C}; and the Jaccard coefficient is used to identify all bounding boxes that delimit the same element, where the intersection area of a pair of bounding boxes divided by the union area of the same bounding boxes must be greater than a Jaccard threshold t_{J}; where the box most likely to enclose the same element is selected by applying the non-maximum suppression process.

Furthermore, the identified element is classified as any of a new oil droplet, a known oil droplet that is on its way to the surface, a known oil droplet that is on the surface, an air bubble or a spurious element.

Specifically, an identified element is detected as a droplet candidate and placed in a first droplet candidate list when the element is not associated with any element already in the first droplet candidate list or in a second droplet candidate list. The element is inserted into the first droplet candidate list with the bounding box of this element and the video frame number where the first identification of this element occurred.

In particular, an element is identified as previously included in the first droplet candidate list, when the bounding box of this element has size equal to and overlaps with the bounding box of an element included in the first droplet candidate list; and wherein the element is included in the first droplet candidate list with the new bounding box information and video frame number where the updated information was collected. The definition of “size equal to or overlaps” is associated with the captured area of the image and the response obtained. That is, the distance between the capture element and the water surface.

More particularly, an element is identified as previously included in the second list of classified as a droplet, when the bounding box of this element is equal in size to and overlaps with the bounding box of an element included in the second list of element classified as a droplet; and wherein the element is included in the second list of classified as a droplet with the new bounding box information and video frame number where the updated information was collected.

In this sense, if an element remains for more than t_{P} seconds in the first droplet candidate list without updating, the element is discarded from the first droplet candidate list; or if it is identified that the total lifetime of the element from the first and last detection of the element is greater than t_{L} seconds, then the element moves to the second list classified as a droplet.

The stage of detecting droplet 6 is performed by an artificial neural network, namely the YOLOv5 network, which receives as input an RGB (red, green, and blue) colored image and outputs bounding boxes for the detected elements as output. The YOLOv5 artificial neural network was pre-trained by its authors with the Coco dataset for all classes predicted in this dataset and was then specialized for the “droplet” class with a dataset created from oily features videos. The hyperparameters used are the same as those considered in pre-training with Coco, carried out by the authors of YOLOv5. Details are provided in the source repository: https://github. com/ultralytics/yolov5.

After the step of processing video 5, this data stored in memory is summarized and stored 9 in a file, such as an XLSX file extension and an MP4 file, in the same folder where the original video in MP4 format was stored.

### Estimating Motion Flow 7

The step of estimating spreading flow 7 is performed through the network used is RIFE HD v3, which receives as input a pair of consecutive color frames (RGB) and produces as output an intermediate color frame (RGB) and two flow maps containing vectors in the two-dimensional image space. The model was pre-trained by its authors with the Vimeo90K dataset. The hyperparameters used were defined by the RIFE authors. Details are provided in the source repository: https://github.com/megvii-research/ECCV2022-RIFE. The artificial neural network used in spreading flow estimation 7 was originally developed for creating intermediate frames in videos with low frames per second rate. In other words, the artificial neural network receives as input two video frames, captured at times t and t+1, and produces an artificial frame with the estimated content for time t+½. In addition to the estimated video frame, the RIFE HD v3 artificial neural network also produces two vector maps or motion flow maps. The first motion flow map indicates where the pixels of the frame at time t went at time t+½. The second motion flow map indicates where the pixels of the frame at time t+1 came from the estimated frame at time t+½.

Specifically, instead of reporting consecutive frames of the video (that is, captured at time t and t+1), according to the method of the present invention, frames captured at time t and t+2 are reported. Therefore, the motion flow maps produced in the present invention are associated in an existing t+1 frame. The portions of these motion flow maps related to the oil on the water surface correspond to the oil droplet spreading estimate.

According to the present invention, a motion flow map is maintained at the same resolution as the video frames. This motion flow map assigns to each pixel of the current frame the identifying number of the droplet that gave rise to the oil present in that pixel (integer value greater than or equal to 1), or the value 0 if the pixel is not associated with the oil in any droplet. The estimate of the spreading flow percentage is given by dividing the number of pixels associated with a droplet by the total number of pixels in the image, 100 times.

More specifically, estimating the motion flow 7 comprises creating an intermediate frame t+1 from two frames of the video captured at time instants t and t+2; and creating two motion flow maps associated with the intermediate frame at time instant t+1, wherein the motion flow maps include vectors in the two-dimensional space of the image; wherein the first motion flow map indicates where the frame pixels at time instant t went at time t+1 and the second motion flow map indicates where the frame pixels at time t+2 came from the intermediate frame at time t+1; and wherein the motion flow maps maintain the same resolution in pixels as the video frames at time instants t and t+2 and assign to each pixel of the frame at time instant t+1 the identifying number of the droplet that gave rise to the oil in that pixel, wherein the identifying number is an integer value greater than or equal to 1, or the value 0 if the pixel is not associated with the oil of any droplet; and wherein the estimate of the percentage of spreading flow is given by dividing the number of pixels associated with a droplet by the total number of pixels in the image, 100 times.

### Tracking of Droplet and Oil Spreading 8

The step of tracking the droplet and oil spreading 8 is described below for ready reference. The pixels in the frame considered as the origin of the oil of a droplet that dissolves on the water surface are those that correspond to the area occupied by the droplet in the last update made by it in the droplet candidate list, before its promotion to the second list of classified as droplets. Therefore, the starting moment is given by this table.

The oil area of each broken droplet is updated frame by frame with the aid of motion flow estimation. This occurs by updating the map that assigns to each pixel of the frame the identifying number of the droplet that gave rise to the oil in that pixel. That is, if the motion flow map indicates that the contents of a pixel with oil at time t have passed to another pixel at time t+1, then the droplet identifier associated with the source pixel is copied to the destination pixel at the map mentioned above.

The area of oil for each droplet (in pixels) is given by counting the number of pixels that contain the droplet identifier on the map in question. The relative area is given by the area of the oil in pixels divided by the total area of the image.

Thus, according to the present invention, tracking the droplet and the spreading the oil 8 comprises defining that the pixels of the frame considered as the origin of the oil of a droplet that dissolves on the water surface are those that correspond to the area occupied by droplet in the last update made to the list of droplet candidates, before its promotion to the second list of those classified as a droplet; wherein the oil area for each droplet in pixels is given by counting the number of pixels that contain the droplet identifier on the map in question; and wherein the relative area is given by the area of the oil in pixels divided by the total area of the image.

### (c) Obtaining Processing Results 10

The user obtains the processing results 10 through the web interface, which activates the Rest API to produce a page that allows download 11 of both the MP4 video initially submitted, and the MP4 video that exhibits the views exemplified in FIG. 1, with marks for identifying detected droplets, tracked droplets and oil spots produced by spreading; and the XLSX file that contains, for each detected droplet, the moment relative to the beginning of the video wherein the beginning of formation of the droplet was detected, the moment at which the droplet disintegrates on the water surface and the relative area occupied by the oil in that droplet at every t seconds of the video during spreading. Furthermore, these results are stored and read from the server 12 HD, available for download.

Therefore, the step of obtaining the processing results 10 includes obtaining the captured video, a video with droplets detected and droplets and spreading tracked; and indication, for each detected droplet, of the moment relative to the beginning of the video wherein the beginning of its formation was detected, the moment in which the droplet disintegrates on the water surface and the relative area occupied by the oil of that droplet every t seconds of the video during spreading.

In this way, objective parameters are provided that characterize the behavior on the water surface of the oil originating from any oily material under study. These parameters allow comparison with any spill history, in order to predict how much faster or slower the intrinsic trend of oil spreading on the surface is, for example. Furthermore, observation is made of how possible mitigating measures, such as retention materials or detergents, for example, would allow expectation of good performance.

### Tracking Indices

Based on the results obtained, tracking indices are implemented. The primary data refers to the percentage of pixels covered on the tracked surface. The results are individualized for each droplet detected and, also, apply to the sum of them, therefore, showing data for the entire diffuse oil. As this representation in pixel ratios is proportional to area ratios, tracking indices are shown in several examples of area ratio applications.

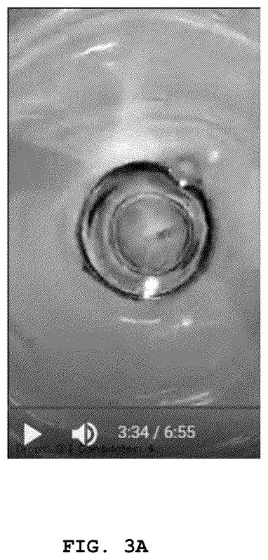

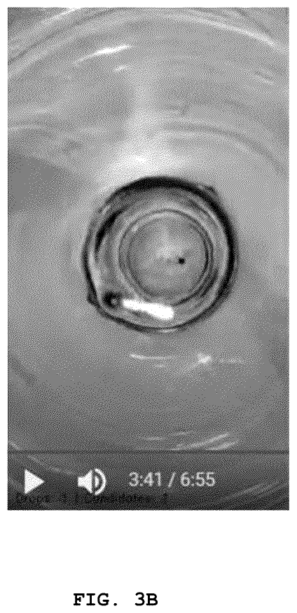

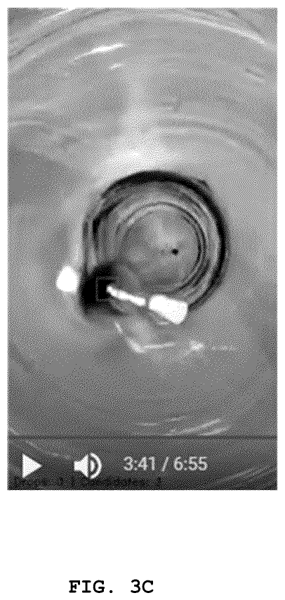

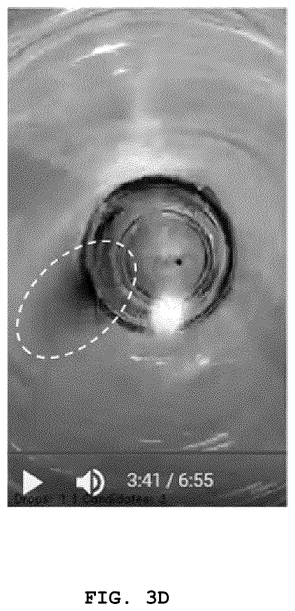

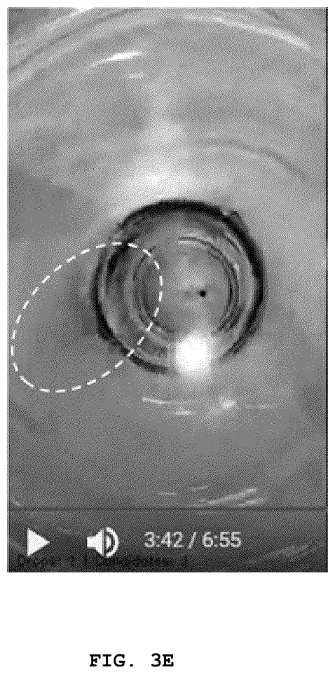

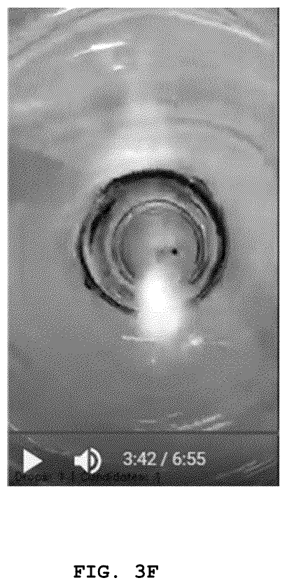

FIGS. 3A, 3B, 3C, 3D, 3E and 3F show an example of application.

FIG. 3A exhibits an image generated showing an experiment with an air bubble, which is correctly not detected as a droplet.

FIG. 3B exhibits an image generated showing the droplet experiment at the moment exactly before the oil expanded onto the surface.

FIG. 3C exhibits an image generated showing the beginning of the oil spreading, with consequent breakup of the droplet.

FIG. 3D exhibits an image generated showing a spot, highlighted by a dashed ellipse, representing oil spreading, but with darkening at the origin of the spot, still indicating the location of the original droplet.

FIG. 3E exhibits an image generated showing a homogeneous spot, highlighted by a dashed ellipse, indicating an advanced state of oil spreading.

FIG. 3F exhibits an image generated showing a surface with the presence of oil that is very difficult to detect by image observation alone, due to its spread already close to total.

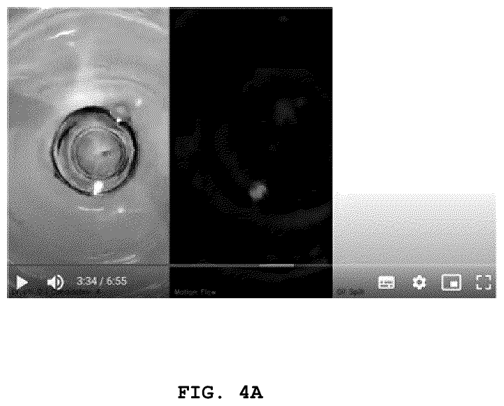

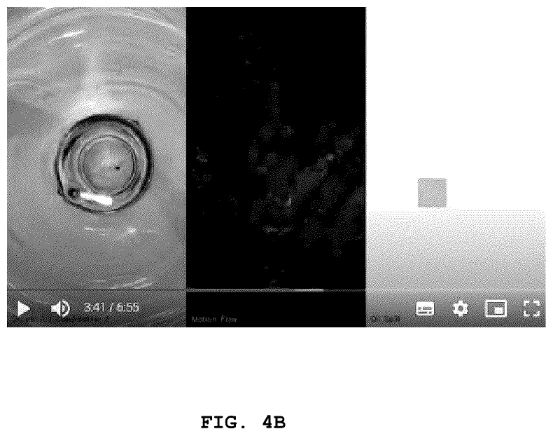

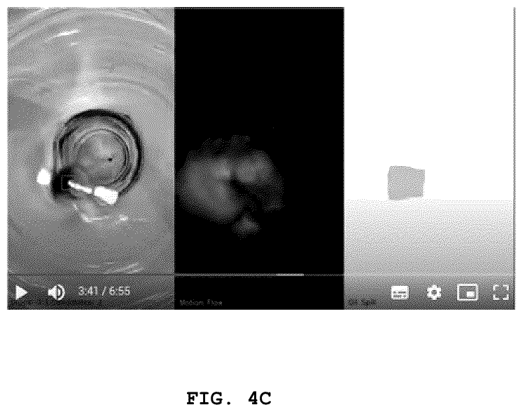

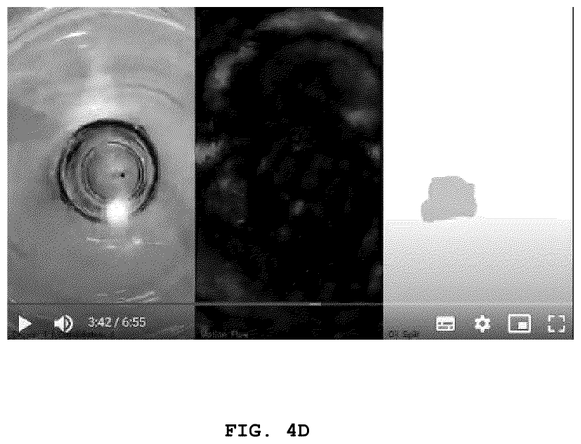

FIGS. 4A, 4B, 4C, 4D and 4E exhibit frames from the sequence that was shown in FIGS. 3A, 3B, 3C, 3D and 3E, respectively, but with processing carried out in accordance with the method of the present invention.

In this sense, according to FIGS. 4A, 4B, 4C, 4D and 4E, the original image is shown as the first image, always on the left; the spread flow frames in the center; and the last image, on the right, contains the response in area of pixels covered by the oil, that is, the processed image.

In FIG. 4A, the image on the right is empty, as there are no pixels assigned to oil.

In FIG. 4B, pixels are assigned, represented by the area of the square that appears in the image on the right.

It is noted that, even in FIG. 4E, wherein the oil is no longer detectable by mere observation with the naked eye (original image on the left), the method proposed in the present invention can still follow its spreading, as shows the image in the center.

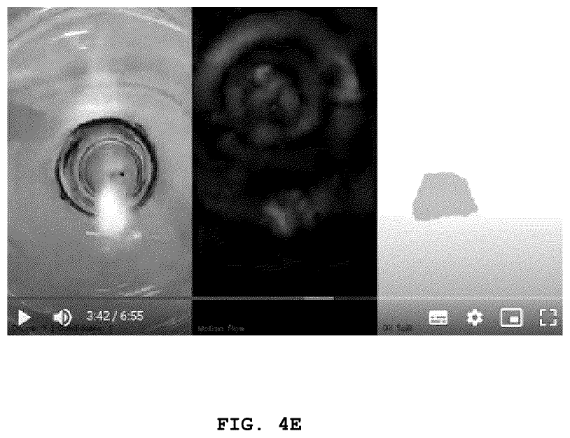

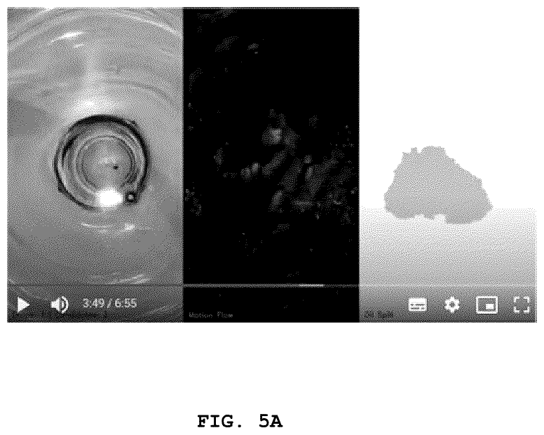

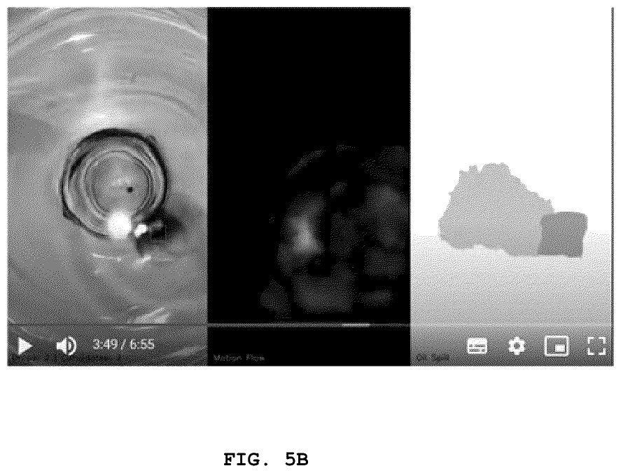

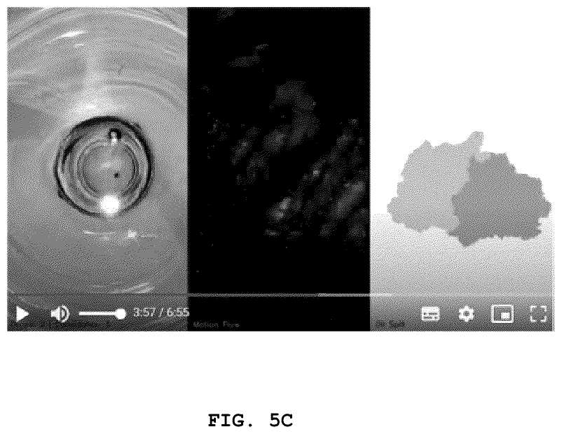

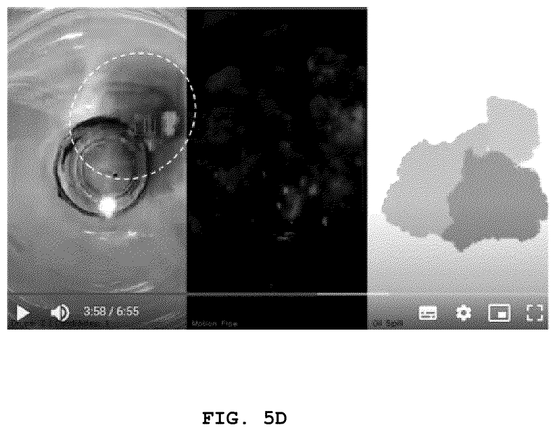

FIGS. 5A, 5B, 5C, 5D, 5E and 5F represent the spreading flow of 3 (three) droplets.

As can be seen in FIG. 5A, which precedes the arrival of the second droplet, it can be seen that the processed image (on the right) shows that there was a follow-up of the first droplet shown in FIG. 4E, because the area of the square in the right frame (referring only to the first droplet) is already considerably larger than in the image on the left of FIG. 4E.

FIG. 5B shows the detection of a second oil droplet, the spread of which appears in orange in the image on the right.

FIG. 5D shows the detection of a third oil droplet, the spread of which appears in beige in the image on the right.

FIG. 5F shows the detection of a fourth droplet, whose spreading appears in green in the image on the right.

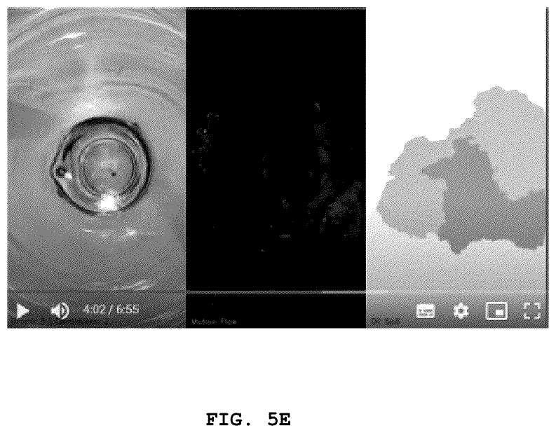

FIG. 6 illustrates the phenomenon of saturation of the water surface by oil from the droplets.

FIG. 6 shows a different situation from the previous ones after the spread of the fourth droplet. Note that the surface allows the presence of oil to be seen well before the fifth droplet hatches. This did not occur in the previous situations and indicates the phenomenon wherein the oil is visibly easily detected by examining the image. This can be called surface saturation, which is the subject of evaluation to better understand the identification of spots on surfaces.

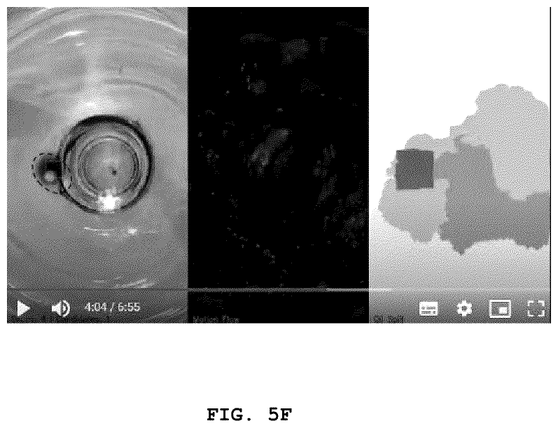

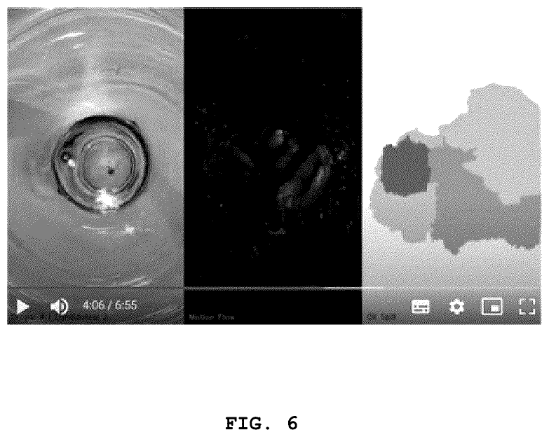

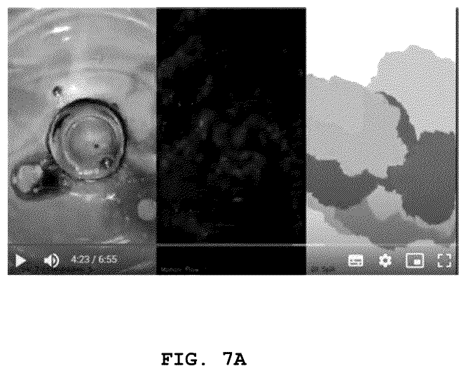

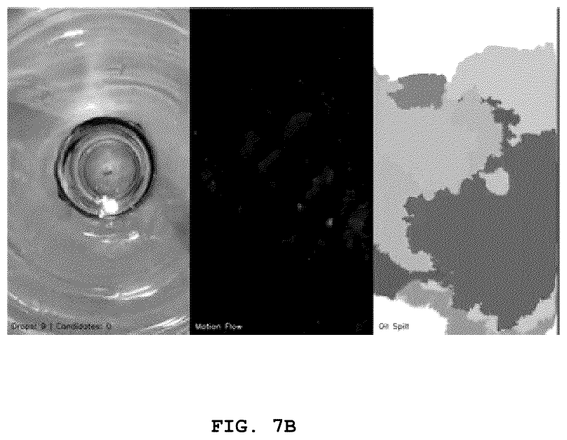

FIG. 7A shows image processing by the method of the present invention, after saturating the surface with 7 droplets.

FIG. 7B shows image processing by the method of the present invention, after saturating the surface with 9 droplets.

### Objective Parameters

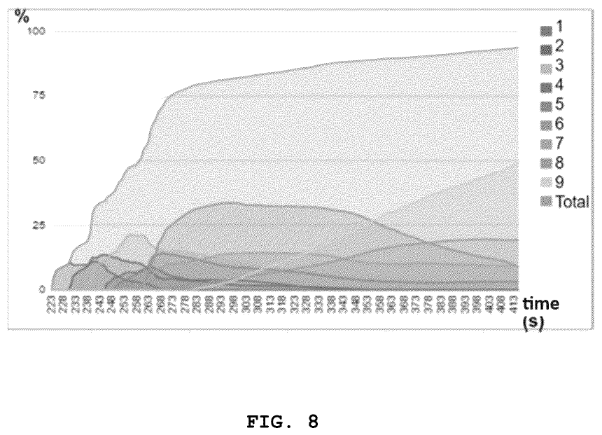

Given that it is considered that the pixel relationships determine each moment of the surface covering by the oil coming from the droplets, the application data also makes it possible to evaluate several aspects related to the phenomenon involved. For example, it is possible to monitor the contributions of droplets to filling the surface with oil, as shown, for example, in FIG. 8.

FIG. 8 displays a graph with the percentage variation in the area (percentage of pixels) of the video processed by the method of the present invention, in FIGS. 3A to 5F, per droplet released and as a function of the shooting time (in seconds).

The parameters, therefore, refer to the individual droplets and the surface coverage. In an initial droplet, it is possible to account for differences in initial spreading and at different times after hatching. This propagation is not linear, and these data can be related to oil properties, if this type of study is considered relevant.

From the moment the oil from a droplet spreads over a region of the surface that was already covered by the oil from a previous droplet, the surface of that previous droplet is considered to begin to decrease. This monitoring of the interaction between the droplets may also be relevant depending on the interests of the studies.

Therefore, below is a list of non-limiting parameters that can be obtained from the data of the method proposed by the present invention:
area covered by the first droplet (free surface) as a function of time;
derivative from the area covered by the first droplet (free surface) in relation to time;
sum of the covered area as a function of time after each droplet hatches;
derivative from the covered area as a function of the time after each droplet hatches;
time until maximum spreading rate per droplet;
derivative of the spreading rate per droplet as a function of time, up to the maximum rate;
percentage of variation in the area covered by oil as a function of time, after releasing the last droplet; and
number of droplets until surface saturation.

Still, according to another preferred embodiment of the present invention, the way the oil spreads or the way the images are captured do not affect the processing of the images. Therefore, the release of oil can be carried out with a pipette at the bottom of a beaker with water, and from this type of images to satellite images of a real spill in a pipeline corrosion event, for example, are processed by the method proposed in the present invention.

Alternatively, the oil release can be done in the laboratory, in a controlled manner, so that the proposed indices can be measured and validated. Too simple release or an actual spill event is too subject to random factors.

Thus, the present invention provides a system for releasing oil, wherein the system releases oil in a controlled manner.

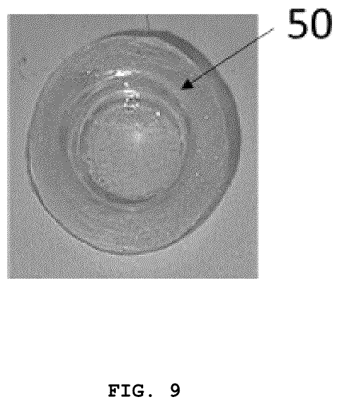

In a preferred embodiment, the system for releasing oil comprises an oleophilic crucible 50, indicated in FIG. 9, which includes a sheet of about 1 mm of plastic material that has an affinity for oil, the sheet is pressed using a blistering and a depression is formed that will contain the oil. The diameter of the crucible 50 is 8 mm and the height from its base to the edge is 3.5 mm (it does not consider the thickness of the edge). The edge thickness varied between 0.1 and 0.2 mm.

Furthermore, in order to obtain physical dimensions of the upward movement of the droplet, a mirror 60 was introduced into the assembly with the crucible 50 in a glass chamber. Mirror 60 received a scale printed on vinyl sheet (waterproof), with subdivisions every 2millimeters.

The glass chamber with mirror 60 has two scales in different colors, with one scale ascending and the other descending. The vinyl paper scales have the height of the crucible as a reference: one starts exactly at this height, the other ends exactly at this height.

Scales can be, for example, 3.6 cm. The distance between the scale bands is 4.2 cm. Experiments range from bottom water down to 1 cm to water down to 3 cm on this scale.

This assembly of the oil release system of the present invention provides, in the same image, the documentation of the surface and the rising droplet. This way, it is not necessary to combine images from different cameras to monitor both images.

The angle of the mirror with the glass wall behind it is 32.3 degrees. The angle with the base (bottom) is 57.7 degrees. With the angle defined, all measurements can be calculated.

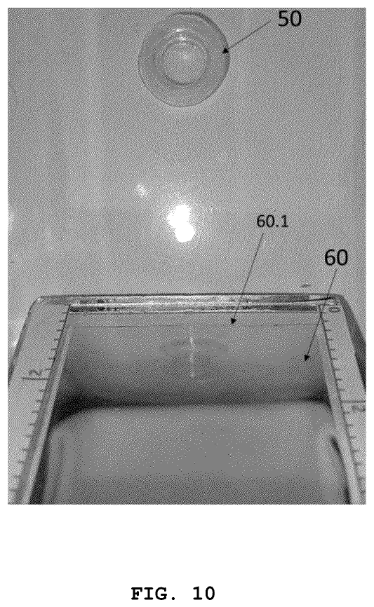

For example, FIG. 10 shows the assembly parts of a preferred embodiment of the oil release system. As can be seen in FIG. 10, a line 60.1 is indicated in red color on the base of the mirror 60. The line 60.1 in red color on the base of the mirror 60 was made by using paint on the side of the crucible and, subsequently, touching the crucible in the mirror. So, the red line shows the exact height of the crucible, and this can be calculated by the image as calibration item. FIG. 10 shows an image that was obtained with the video capture device camera positioned horizontally, that is, in a parallel position to the water surface and a support bench.

The crucible 50 is filled with oil and the mirror chamber 60 is filled with water. The oil tends to rise, due to the difference in density, but it does not do so directly from the depression, since as the material of the crucible 50 is oleophilic, the oil drags along the edge until it is released from it. This provides a well-defined droplet formation.

Thus, according to a preferred embodiment of the present invention, the oil releasing system comprises an oleophilic crucible 50 with a sheet of plastic material having a depression for containing oil; a camera with a mirror 60; and a video capture device, wherein the video capture device is positioned parallel to the water surface, fixed on a base; wherein the video captured by the video capture device is processed in accordance with the method for measuring the oil dispersion capacity on the water surface of the present invention. Furthermore, the chamber is made of glass and has two measuring scales; and the mirror comprises a physical indication for calibration.

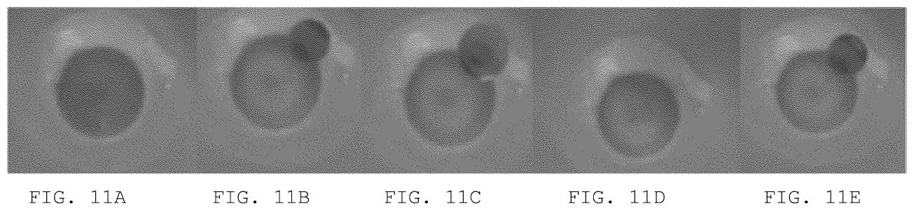

FIGS. 11A, 11B, 11C, 11D and 11E show droplet formation in an example of an application monitored by a cell phone camera.

FIG. 11A shows crucible 50 filled with oil.

FIG. 11B shows the moment of releasing the droplet from the edge.

FIG. 11C shows the droplet reaching the surface, wherein the droplet appears larger because it is closer to the cell phone camera.

FIG. 11D shows the moment when the oil began to spread on the surface.

FIG. 11E shows the release of a new droplet, wherein the reproducibility of the droplet volume at the moment of release is noted in comparison with FIG. 11B.

The oil release system of the present invention allows the exact detection of the moment of releasing the droplet from the wall of the crucible 50. As the time is measured on the film itself and the height is known from the water level in the image, the exact time for the upward movement of the droplet can be calculated and any calculation methodology can be calibrated.

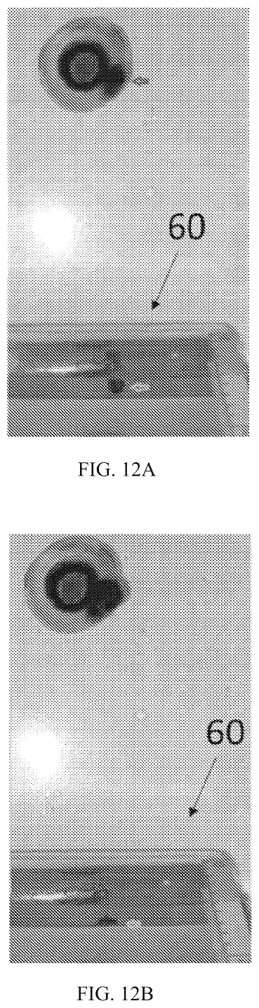

FIG. 12A shows the moment of releasing the droplet from the edge of the crucible 50, where the water level is highlighted with the red arrow; the top view of the droplet on the edge is highlighted with the blue arrow; and the side view through mirror 60 of the upward moving droplet is highlighted with the green arrow.

FIG. 12B shows the moment when the released droplet hits the surface. The side view through mirror 60 of the upward moving droplet is highlighted with the green arrow.

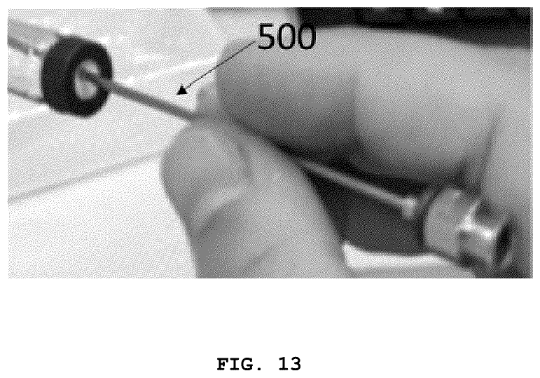

According to another preferred embodiment, the present invention defines a system for releasing oil comprising a hollow L-shaped needle 500, wherein the needle 500 is illustrated in FIG. 13.

Specifically, the 500 L hollow needle allows oil to flow through its hollow center, as in a syringe needle for vaccine or drug injections.

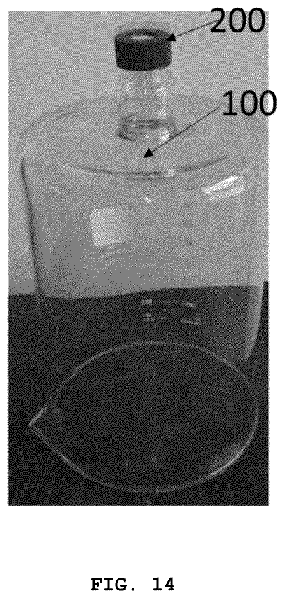

Furthermore, a container 100 is adapted to receive this needle 500, as the container comprises a rubber septum inlet 200, wherein the needle is received through the rubber septum inlet 200, as shown in FIG. 14. The needle 500 pierces the septum 200, which, being made of very resilient rubber, quickly compresses the metal of the needle 500, and this compression seals the hole in the septum 200.

The advantage of using a 500 L hollow needle is the use of large volumes of water and generation of larger droplets, unlike the assembly of the oleophilic crucible 50.

Therefore, in the assembly with an oleophilic crucible 50, small droplets are observed traveling upwards at small heights, so that there is clarity in the images with these small dimensions.

When mounting with a 500 L hollow needle, larger droplets are observed at greater heights, even with droplet registration clarity, and these relatively large dimensions do not allow for adequate framing of the mirror 60 and/or scale in the images.

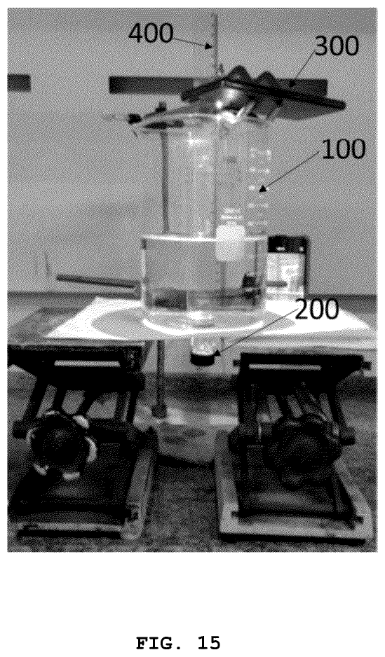

FIG. 15 represents the system for releasing oil according to a preferred embodiment of the present invention, comprising a container 100 with a rubber septum 200, wherein the container 100 is oriented so that the septum (200) is arranged with its inlet downwards, at the bottom of the oil release system; a video capture device 300 movably arranged on the top of the oil release system; a 400 measuring scale; and a 500 L hollow needle.

More specifically, the video capture device 300 can be a cell phone or a tablet, for example.

Thus, according to a preferred embodiment, the oil releasing system of the present invention comprises a container 100 with a septum 200, wherein the container 100 is oriented so that the septum 200 is arranged with its inlet down; a hollow needle 500, wherein the hollow needle 500 is inserted into the septum 200; a video capture device 300, wherein the video capture device 300 is movably arranged on the top of the container 100; and a measuring scale 400; wherein the video captured by the video capture device 300 is processed in accordance with the method for measuring the oil dispersion capacity on a water surface of the present invention. Furthermore, the video capture device 300 is movably arranged on the top of the container 100 via a support.

More specifically, in FIG. 15, an experiment with seawater included in container 100 is represented.

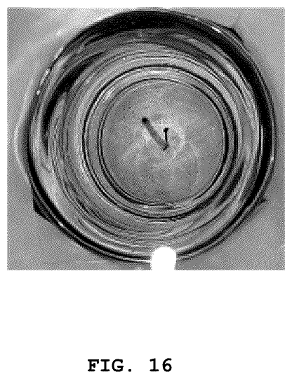

FIG. 16 is an image captured by the video capture device 300, positioned at the top of the oil release system of the present invention and shows the needle 500 inserted into the septum 200 of the container 100. FIG. 16 shows the variation in height and the position of the tip of the needle 500 at the oil release point.

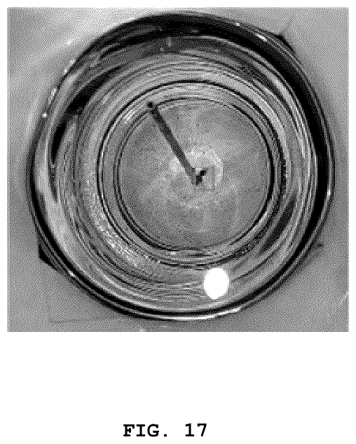

FIG. 17 shows another variation in the height of the tip of the needle 500 inserted into the septum 200 of the container 100.

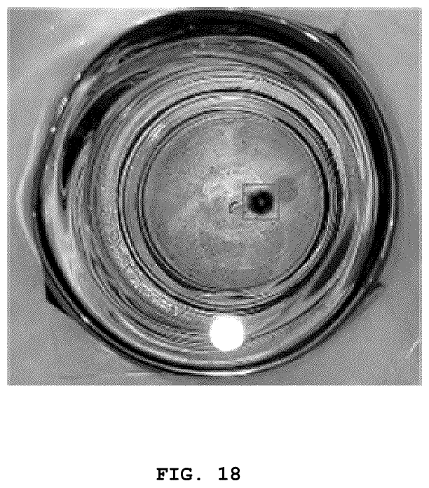

FIG. 18 shows the release of an oil droplet into seawater from the tip of needle 500, wherein the droplet is highlighted with a purple rectangle.

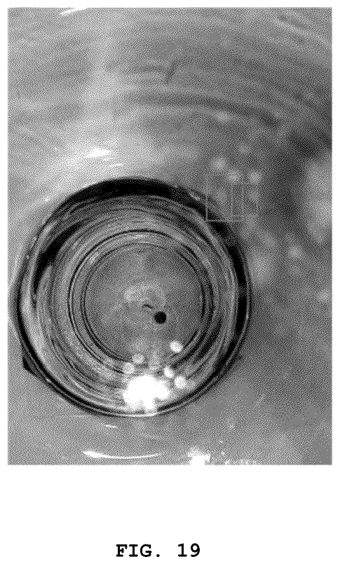

FIG. 19 illustrates a droplet that reaches the surface and spreads, while a new droplet is already ready at the tip of the needle 500 to be released, when observation of the previous droplet ceases.

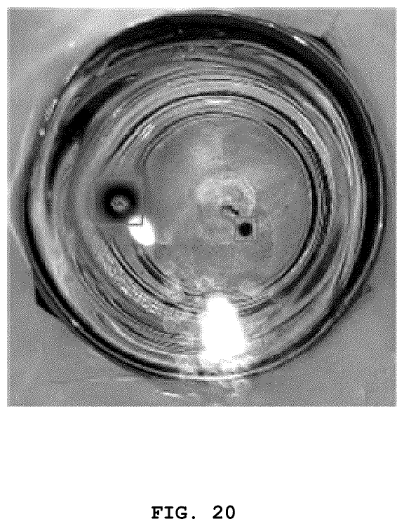

FIG. 20, following FIG. 19, illustrates when a new droplet is released (larger square in purple), the surface has no visible traces of the previous droplet, but the motion flow allows following the spreading of the previous droplet on the surface. Additionally, the smallest square in purple shows the next droplet to be released.

Those skilled in the art will value the knowledge shown here and will be able to reproduce the invention in the embodiments and in other variants, covered within the scope of the attached claims.

1. A method for measuring the oil dispersion capacity on a water surface comprising the steps of:
capturing at least one video;
processing the video, which comprises identifying at least one frame-by-frame element of the video, independently between frames; wherein the at least one element is identified by at least one bounding box; further including the step of droplet detection, wherein the element is detected as a droplet candidate element, or an element classified as a droplet;
estimating the motion flow, which comprises creating an intermediate frame t+1 from two frames of the video captured at time instants t and t+2; and creating two motion flow maps associated with the intermediate frame at time instant t+1, wherein the motion flow maps include vectors in the two-dimensional space of the image; wherein the first motion flow map indicates where the frame pixels at time instant t went at time instant t+1 and the second motion flow map indicates where the frame pixels at time instant t+2 came from the intermediate frame at time instant t+1; and
wherein the motion flow maps maintain the same resolution in pixels as the video frames at time instants t and t+2 and assign to each pixel of the frame at time instant t+1 the identifying number of the droplet that gave rise to the droplet oil present in that pixel, where the identifying number is an integer value greater than or equal to 1, or the value 0 if the pixel is not associated with the oil of any droplet; and wherein the estimate of the percentage of spreading flux is given by dividing the number of pixels associated with a droplet by the total number of pixels in the image, 100 times;
tracking the droplet and the spreading of the oil comprises defining that the pixels in the frame considered as the origin of the oil from a droplet that dissolves on the water surface are those that correspond to the area occupied by the droplet in the last update made by it in the list of droplet candidates, before their promotion to the second list of those classified as droplet; wherein the oil area for each droplet in pixels is given by counting the number of pixels that contain the droplet identifier on the map in question; and wherein the relative area is given by the area of the oil in pixels divided by the total area of the image; and
obtaining the results of processing, which includes obtaining the captured video, a video with detected droplets and tracked droplets and spreading; and indication, for each detected droplet, of the moment relative to the beginning of the video wherein the beginning of its formation was detected, the moment wherein the droplet disintegrates on the water surface and the relative area occupied by the oil of that droplet every t seconds of the video during spreading.

2. The method according to claim 1, wherein the at least one video is captured by at least one capture device comprising at least one camera.

3. The method according to claim 1, wherein in the step of video processing, a bounding box is discarded when the confidence in detection is lower than a temporal threshold (t_{C}).

4. The method according to claim 1, characterized in that wherein in the step of video processing, the Jaccard coefficient is used to identify all the bounding boxes that delimit the same element, wherein the intersection area of a pair of bounding boxes divided by the area of union of the same bounding boxes must be greater than a Jaccard threshold (t_{J}).

5. The method according to claim 1, wherein the box most likely to delimit the same element is selected by applying the non-maximum suppression process.

6. The method according to claim 1, wherein the identified element is classified as any of: a new oil droplet, an already known oil droplet that is on its way to the surface, an already known oil droplet and that is on the surface, an air bubble or a spurious element.

7. The method according to claim 1, wherein an identified element is detected as a droplet candidate and inserted into a first droplet candidate list when the element is not associated with any element already present in the first droplet candidate list or on a second list of classified as droplet.

8. The method according to claim 7, wherein the element is inserted into the first droplet candidate list with the bounding box of this element and the number of the video frame where the first identification of this element occurred.

9. The method according to claim 1, wherein an element is identified as previously included in the first droplet candidate list, when the bounding box of this element has a size equal to and overlaps with the bounding box of an element included in the first droplet candidate list; and wherein the element is included in the first droplet candidate list with the new bounding box information and video frame number where the updated information was collected.

10. The method according to claim 1, wherein an element is identified as previously included in the second list of classified as a droplet, when the bounding box of this element has a size equal to and overlaps with the bounding box of an element included in the second list of elements classified as droplet; and wherein the element is included in the second list of classified as droplet with the new bounding box information and video frame number where the updated information was collected.

11. The method according to claim 1, wherein if an element remains for more than t_{P} seconds in the first droplet candidate list without updating, the element is discarded from the first droplet candidate list; or if it is identified that the total lifetime of the element from the first and last detection of the element is greater than t_{L} seconds, then the element moves to the second list of classified as a droplet.

12. The method according to claim 1, wherein t_{P} and t_{L} depend on the oil injection flow rate and the desired interval between the arrangement of droplets, ranging from 1 to 10 seconds.

13. An oil release system comprising:
an oleophilic crucible with a sheet of plastic material with a depression for containing oil,
a camera with a mirror; and
a video capture device, wherein the video capture device is positioned parallel to the water surface, fixedly on a base;
wherein the video captured by the video capture device is processed in accordance with the method for measuring the oil dispersion capacity on a water surface defined in claim 1.

14. A system according to claim 12, wherein the chamber is made of glass and has two measuring scales.

15. A system according to claim 12, wherein the mirror comprises a physical indication for calibration.

16. An oil release system comprising:
a container with a septum, wherein the container is oriented so that the septum is arranged with its inlet downward;
a hollow needle, wherein the hollow needle is inserted into the septum;
a video capture device, wherein the video capture device is movably arranged on the top of the container; and
a measuring scale;
wherein the video captured by the video capture device is processed in accordance with the method for measuring the oil dispersion capacity on a water surface defined in claim 1.

17. The system according to claim 12, wherein the video capture device is movably arranged in the upper part of the container through a support.

18. A computer-readable storage media characterized by comprising, stored within itself, a set of computer-readable instructions, which when executed by one or more computers, the one or more computers performs the method for measuring the oil dispersion capacity on a water surface, as defined in claim 1.
